(19) United States
(12) Patent Application Publication
(10) Pub. No.: US 2010/0174143 A1
(43) Pub. Date: Jul. 8, 2010

(54) DUAL SEAL WITH BELLOWS

(75) Inventors: Michael E. Abrams, New York, NY (US); Christopher A. Battles, Seymour, CT (US); Paul DiCesare, Easton, CT (US); Danial P. Ferreira, Milford, CT (US); Gregory R. Hires, Fairfield, CT (US)

Correspondence Address: Tyco Healthcare Group LP, 60 MIDDLETOWN AVENUE, NORTH HAVEN, CT, 06473, US

(73) Assignee: Tyco Healthcare Group LP

(21) Appl. No.: 12/628,609

(22) Filed: Dec. 1, 2009

Related U.S. Application Data
(60) Provisional application No. 61/142,650, filed on Jan. 6, 2009.

(51) Int. Cl. A61B 1/00

(52) U.S. Cl. 600/121

(57) ABSTRACT

A seal assembly which includes a housing defining a longitudinal axis, a proximal portion and a distal portion and including a bellows disposed along a length thereof. A first sealing membrane and a second sealing membrane associated with the proximal portion of the housing, each sealing membrane configured to create a substantially fluid-tight seal around a surgical instrument inserted therethrough. The bellows configured to allow at least a portion of the housing to move radially with respect to the longitudinal axis.

### CROSS REFERENCE TO RELATED APPLICATION

The present application claims the benefit of and priority to U.S. Provisional Application Ser. No. 61/142,650 filed on Jan. 6, 2009, the entire contents of which are incorporated herein by reference.

### BACKGROUND

1. Technical Field

The present disclosure relates to a seal system which is adapted to allow the introduction of surgical instrumentation into a patient's body. In particular, the present disclosure is applicable to a cannula assembly wherein a cannula housing includes or is adapted to receive a seal assembly to sealingly accommodate instruments of different diameters inserted through the seal assembly and cannula.

2. Description of the Related Art

In laparoscopic procedures surgery is performed in the interior of the abdomen through a small incision; in endoscopic procedures surgery is performed in any hollow viscous of the body through narrow tubes or cannula inserted through a small entrance incision in the skin. Laparoscopic and endoscopic procedures generally require that any instrumentation inserted into the body be sealed, i.e. provisions must be made to ensure that gases do not enter or exit the body through the incision as, for example, in surgical procedures in which the surgical region is insufflated. Moreover, laparoscopic and endoscopic procedures often require the surgeon to act on organs, tissues, and vessels far removed from the incision, thereby requiring that any instruments used in such procedures be relatively long and narrow.

For such procedures, the introduction of a tube into certain anatomical cavities such as the abdominal cavity is usually accomplished by use of a trocar assembly comprised of a cannula assembly and an obturator assembly. Since the cannula assembly provides a direct passage for surgical instrumentation from outside the patient's body to access internal organs and tissue, it is important that the cannula assembly maintain a relatively gas-tight interface between the abdominal cavity and the outside atmosphere. The cannula assembly thus generally includes a cannula attached to a cannula housing containing a seal assembly adapted to maintain a seal across the opening of the cannula housing.

Since surgical procedures in the abdominal cavity of the body require insufflating gases to raise the cavity wall away from vital organs, the procedure is usually initiated by use of a Verres needle through which a gas such as CO₂ is introduced into the body cavity, thereby creating a pneumoperitoneum. Thereafter, the obturator of the obturator assembly is inserted into the cannula assembly and used to puncture the abdominal wall. The gas provides a positive pressure which raises the inner body wall away from internal organs, thereby providing the surgeon with a region within which to operate, avoiding unnecessary contact with the organs by the instruments inserted through the cannula assembly. Following removal of the obturator assembly from the cannula assembly, laparoscopic or endoscopic surgical instruments may be inserted through the cannula assembly to perform surgery within the abdominal cavity.

Without the obturator assembly to block the flow of insufflation gas out from the cavity, other structure must be provided to maintain a relatively fluid-tight interface between the abdominal cavity and the outside atmosphere. Generally in the context of insufflatory surgical procedures, there are two sealing requirements for cannula assemblies. The first requirement is to provide a substantially fluid-tight seal when an instrument is not being introduced into or is not already present in the cannula. The second requirement is to provide a substantially fluid-tight seal when an instrument is being introduced into or is already present in the cannula. Additionally, as endoscopic and laparoscopic surgical procedures and techniques have advanced, it has become desirable to accommodate surgical instrumentation of varying outside diameters through a single cannula assembly in a given surgical procedure, thereby minimizing the number of cannulae required and facilitating efficiency in the surgical procedure.

### SUMMARY

In accordance with the present disclosure a seal assembly is provided which includes a housing defining a longitudinal axis and having a proximal portion and a distal portion. The housing includes a bellows disposed along a length thereof. A first sealing membrane and a second sealing membrane associated with the proximal portion of the housing are provided. Each sealing membrane is configured to create a substantially fluid-tight seal around a surgical instrument inserted therethrough. The bellows is configured to allow at least a portion of the housing to move radially with respect to the longitudinal axis. The bellows may be configured to allow the proximal portion of the housing to move longitudinally with respect to the distal portion of the housing.

In disclosed embodiments, a distal portion of the housing is configured to mechanically engage a cannula assembly. At least one of the housing and the cannula assembly may include a sealing mechanism which is configured and dimensioned to provide a substantially fluid-tight seal in the absence of a surgical instrument passing therethrough.

The first sealing membrane may be larger than the second sealing membrane. The first sealing membrane may be adapted to receive a surgical instrument having a diameter in the range of about 8 mm to about 15 mm and/or the second sealing membrane may be adapted to receive a surgical instrument having a diameter in the range of about 4 mm to about 8 mm.

In disclosed embodiments, at least one of the first sealing membrane and the second sealing membrane is disposed in a valve. The valves may be dome valves.

A method of introducing surgical instrumentation into a patient's body is also provided, the method including the step of providing a seal assembly including a housing defining a longitudinal axis, the housing having a proximal portion and a distal portion and including a bellows disposed along a length thereof, and a first sealing membrane and a second sealing membrane associated with the proximal portion of the housing, each sealing membrane configured to create a substantially fluid-tight seal around a surgical instrument inserted therethrough. The method further includes the steps of moving the proximal portion of the housing with respect to the distal portion of the housing and inserting a first surgical instrument through the first sealing membrane such that at least a portion of the first surgical instrument is adjacent tissue.

In disclosed embodiments, the method includes the step of inserting a second surgical instrument through the second sealing membrane such that at least a portion of the second surgical instrument is adjacent tissue. The step of removing the first surgical instrument may also be included.

The step of mechanically engaging the seal assembly with a cannula assembly may also be included. The proximal portion of the housing may be moved with respect to the distal portion of the housing in a radial direction.

### BRIEF DESCRIPTION OF THE DRAWINGS

Various embodiments of the present disclosure are described herein with reference to the drawings wherein:

FIG. 1 is a longitudinal cross-sectional view of the seal assembly.

FIG. 2 is an exploded view of a cannula assembly, showing a duckbill valve fitting therein.

FIG. 3 is a longitudinal cross-sectional view of the seal assembly mounted on the cannula assembly with a surgical instrument passing therethrough.

FIG. 4. is a perspective view of the seal assembly.

### DETAILED DESCRIPTION

The seal assembly of the present disclosure, either alone or in combination with a cannula assembly, provides a substantial seal between a body cavity of a patient and the outside atmosphere before, during and after insertion of an instrument through the seal assembly. The seal assembly of the present disclosure is capable of accommodating instruments of varying diameters, e.g., from about 4 mm to about 15 mm, by providing a fluid-tight seal around each instrument when inserted through either the first valve or the second valve.

The seal assembly of the present disclosure contemplates the introduction and manipulation of various types of instrumentation adapted for insertion through a trocar and/or cannula assembly while maintaining a substantially fluid-tight interface about the instrument to preserve the atmospheric integrity of a surgical procedure from gas and/or fluid leakage. Specifically, the seal assembly incorporates a radially and longitudinally deflectable seal housing which accommodates manipulation of the surgical instrument therein. This feature of the present disclosure helps minimize the entry and exit of gases and/or fluids to/from the body cavity during manipulation of the instrumentation. Examples of instrumentation include, but are not limited to, clip appliers, graspers, dissectors, retractors, staplers, laser probes, photographic devices, endoscopes and laparoscopes, tubes, and the like. Such instruments will collectively be referred to as “instruments” or “instrumentation”.

In the following description, as is traditional, the term “proximal” refers to the portion of the device closer to the operator while the term “distal” refers to the portion of the device farther from the operator.

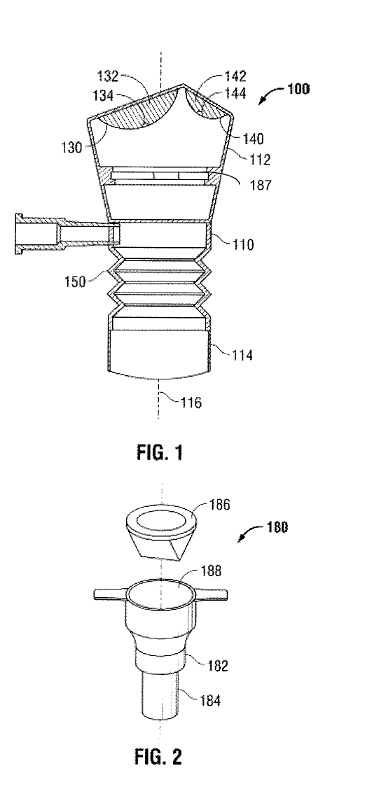

Referring now to the drawings, FIG. 1 illustrates an access port or seal assembly 100 including housing 110 defining longitudinal axis 116. Housing 110 has proximal portion 112 and distal portion 114. Housing 110 includes a bellows 150 disposed along a length thereof. Bellows 150 is fabricated from a resilient material, e.g., isoprene, to facilitate the movements described hereinbelow. First valve 130 is disposed at proximal portion 112 of housing 110. First valve 130 (e.g., a dome valve) includes sealing membrane 132. An instrument seal 134 is defined within sealing membrane 132 to allow surgical instrumentation to pass therethrough. In disclosed embodiments, first valve 130, sealing membrane 132 and instrument seal 134 are dimensioned and configured for receiving instruments having a diameter in the range of about 8 mm to about 15 mm. Sealing membrane 132 is configured to create a substantially fluid-tight seal around surgical instrumentation disposed therein. Similarly, second valve 140 includes sealing membrane 142. An instrument seal 144 is defined within sealing membrane 142 to allow relatively small surgical instrumentation to pass therethrough. Second valve 140 may be a dome valve. In disclosed embodiments, second valve 140, sealing membrane 142 and instrument seal 144 are dimensioned and configured for receiving instruments having a diameter in the range of about 4 mm to about 8 mm. Sealing membrane 142 is configured to create a substantially fluid-tight seal around surgical instrumentation disposed therein.

In a disclosed embodiment, seal assembly 100 is detachably mounted to cannula assembly 180. As shown in FIG. 2, cannula assembly 180 includes cannula housing 182 and cannula 184 which is attached thereto and which extends distally therefrom. In disclosed embodiments, cannula assembly 180 includes a sealing mechanism, such as distally directed duckbill valve 186 mounted in interior region 188 of cannula housing 182. Duckbill valve 186 provides a substantially fluid-tight seal when communicating with an insufflated body cavity to substantially prevent escape of gases and fluids from inside the body cavity when no instrument is present in cannula assembly 180. It is also envisioned that seal assembly 100 includes a substantially fluid-tight seal 187 (FIG. 1) which provides a substantially fluid-tight seal along with or in alternative to duckbill valve 186. Dual seal assembly 100 may be associated with, or joined to, cannula assembly 180 in a variety of ways. In a disclosed embodiment, seal housing 110 of seal assembly 100 and cannula housing 182 of cannula assembly 180 are adapted to detachably engage each other, e.g., through a bayonet lock, threaded connection, or like mechanical means.

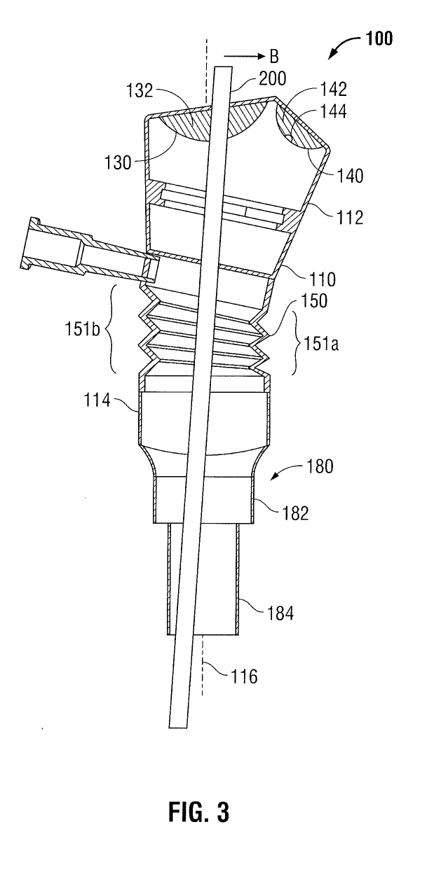

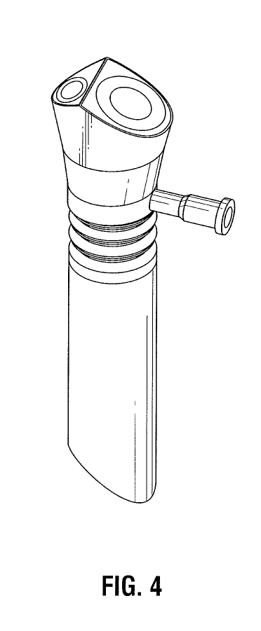

The operation of dual seal assembly 100 in conjunction with cannula assembly 180 will now be described with reference to FIGS. 1 and 3. Prior to insertion of a surgical instrument, duckbill valve 186 provides a fluid-tight seal within cannula assembly 180. FIG. 1 illustrates dual seal assembly 100 in a first or at-rest position in which bellows 150 is substantially symmetrical about longitudinal axis 116. Seal assembly 100 may be mounted on cannula assembly 180. In this position, seal assembly 100 can receive surgical instrumentation through first valve 130 or second valve 140, depending on the diameter of the instrument. First valve 130 is shown as a relatively large dome valve and is configured to receive instrumentation having a diameter in the range of about 8 mm to about 15 mm. Second valve 140 is shown as a relatively small dome valve and is configured to receive instrumentation having a diameter in the range of about 4 mm to 8 mm.

An instrument is inserted into seal assembly 100 through instrument seal 134 or 144, depending on the diameter of the instrument. Seal membranes 132 and 142, which define instrument seals 134 and 144, respectively, stretch to accommodate the instrument diameter passing therethrough, as necessary. The instrument 200 is advanced through instrument seal 134 or 144, whereby a seal is created between sealing membrane 132 or 134 and the instrument passing therethrough. The instrument 200 is further advanced distally through housing 110, cannula assembly 180, duckbill valve 186 and into cannula 184.

Once instrumentation is positioned as described above, via first valve 130 or second valve 140, it may be desired to move the instrument in order to perform surgical procedures. As shown in FIG. 3, when a surgeon moves instrument 200 radially in the direction of arrow B, away from longitudinal axis 116, bellows 150 is compressed along the edge 151a facing the direction of B and elongated along the edge 151b facing away from the direction of B. This above described action allows the proximal portion 112 of housing 110 to move radially with respect to respect to longitudinal axis 116. Distal portion 114 of housing 110 remains relatively stationary, thereby maintaining the connection between cannula assembly 180 and housing 110. Proximal portion 112 of housing 110 may also be moved longitudinally with respect to distal portion 114 of housing 110, thereby maintaining the seal around surgical instrumentation disposed therein. Allowing proximal portion 112 of seal assembly 100 to move in conjunction with instrument 200 maintains the integrity of the seal around instrumentation passing therethrough. Instrumentation inserted through seal assembly 100 may ultimately be removed from seal assembly 100 by pulling the instrumentation proximally until the entire instrument is removed from seal assembly 100.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A seal assembly comprising:
a housing defining a longitudinal axis, the housing having a proximal portion and a distal portion and including a bellows disposed along a length thereof; and
a first sealing membrane and a second sealing membrane associated with the proximal portion of the housing, each sealing membrane configured to create a substantially fluid-tight seal around a surgical instrument inserted therethrough;
wherein the bellows is configured to allow at least a portion of the housing to move radially with respect to the longitudinal axis.

2. The seal assembly of claim 1, wherein the distal portion of the housing is configured to mechanically engage a cannula assembly.

3. The seal assembly of claim 2, wherein at least one of the housing and the cannula assembly includes a sealing mechanism which is configured and dimensioned to provide a substantially fluid-tight seal in the absence of a surgical instrument passing therethrough.

4. The seal assembly of claim 1, wherein the first sealing membrane is larger than the second sealing membrane.

5. The seal assembly of claim 1, wherein the first sealing membrane is adapted to receive a surgical instrument having a diameter in the range of about 8 mm to about 15 mm.

6. The seal assembly of claim 1, wherein the second sealing membrane is adapted to receive a surgical instrument having a diameter in the range of about 4 mm to about 8 mm.

7. The seal assembly of claim 5, wherein the second sealing membrane is adapted to receive a surgical instrument having a diameter in the range of about 4 mm to about 8 mm.

8. The seal assembly of claim 1, wherein at least one of the first sealing membrane and the second sealing membrane is disposed in a valve.

9. The seal assembly of claim 8, wherein at least one valve is a dome valve.

10. The seal assembly of claim 1, wherein the bellows is configured to allow the proximal portion of the housing to move longitudinally with respect to the distal portion of the housing.

11. A method of introducing surgical instrumentation into a patient's body comprising the steps of:
providing a seal assembly, the seal assembly comprising:
a housing defining a longitudinal axis, the housing having a proximal portion and a distal portion and including a bellows disposed along a length thereof, and a first sealing membrane and a second sealing membrane associated with the proximal portion of the housing, each sealing membrane configured to create a substantially fluid-tight seal around a surgical instrument inserted therethrough;
moving the proximal portion of the housing with respect to the distal portion of the housing; and
inserting a first surgical instrument through the first sealing membrane such that at least a portion of the first surgical instrument is adjacent tissue.

12. The method of claim 11, further comprising the step of inserting a second surgical instrument through the second sealing membrane such that at least a portion of the second surgical instrument is adjacent tissue.

13. The method of claim 11, further comprising the step of removing the first surgical instrument.

14. The method of claim 11, further comprising the step of mechanically engaging the seal assembly with a cannula assembly.

15. The method of claim 11, wherein the proximal portion of the housing is moved with respect to the distal portion of the housing in a radial direction.
